(19) United States
(12) Patent Application Publication
(10) Pub. No.: US 2023/0174415 A1
(43) Pub. Date: Jun. 8, 2023

(54) METHOD FOR PRODUCING AN IMPLANT FOR INSERTING INTO AN EYE, IN PARTICULAR FOR INSERTING INTO THE SCHLEMM'S CANAL OF AN EYE

(71) Applicant: aixtent GmbH, Alsdorf (DE)

(72) Inventors: Reinhart Poprawe, Aachen (DE); Axel von Wallfeld, Herzogenrath (DE); Uwe Clasen, Aachen (DE)

(21) Appl. No.: 17/917,457

(22) PCT Filed: Mar. 30, 2021
(86) PCT No.: PCT/DE2021/000057

§ 371 Date: Oct. 6, 2022

(30) Foreign Application Priority Data
Apr. 9, 2020 (DE) 10 2020 002 231.6

(51) Int. Cl. C03C 15/00; A61F 9/007; A61L 31/02; C03C 23/00

(52) U.S. Cl. CPC C03C 15/00; A61F 9/00781; A61L 31/026; C03C 23/0025; A61F 2240/001

(57) ABSTRACT

Exemplary arrangements relate to a method for producing an implant, in particular an implant configured to be inserted into a Schlemm's canal of an eye, which includes the steps of providing an implant blank, which implant blank is comprised of material that is permeable to laser radiation. The method further includes subjecting at least one region of the implant blank to laser radiation. Subsequent to subjecting the least one region to radiation, the method further includes removing material from the at least one region via fluid etching.

### TECHNICAL FIELD

Exemplary arrangements relate to a method for producing an implant, in particular for inserting into an eye and in particular for inserting into the Schlemm's canal of an eye. Exemplary arrangements further relate to an implant produced according to this method.

### BACKGROUND

Implants for insertion in the Schlemm's canal are known for example from WO 2010/072574. Such implants and the procedures for insertion thereof may benefit from improvements.

### SUMMARY

In use of an implant configured for insertion in the Schlemm's canal of an eye, the circular Schlemm's canal is exposed primarily at one location, and is gently expanded circularly with an inserted and flexibly designed microcatheter. At the same time or subsequently, a high-molecular-weight viscoelastic substance is injected. The exemplary implant which can be introduced into the lumen of the Schlemm's canal consists of a flexible, elongated tube with multiple openings or holes that communicate with an interior space. It may have at least one connecting part that is continuously oriented in the axial direction and with an arched surface, and in the inserted state is arranged to lie against the inner wall of the lumen for support. The individual openings and holes form a direct connection which is kept permanently open between the trabecular meshwork and the individual small collector channels of the distal outflow system, so that the natural transtrabecular outflow of the aqueous humor through the episcleral venous system into the blood circulation is assured.

In a healthy eye, the circulating aqueous humor (humor aquosus) drains from the posterior chamber to the anterior chamber and is discharged into the Schlemm's canal via the trabecular meshwork in the chamber angle (angulus iridocornealis), from there it is delivered into the blood circulation through the episcleral venous system. In pathological conditions of the eye, in particular when resistances occur due to closure of the Schlemm's canal caused for example by adhesion or the like, a continuous drainage of the aqueous humor, which is produced by the epithelium of the ciliary body and is constantly being renewed, is often no longer assured adequately, or even not at all. This can cause the intraocular pressure (IOP) to rise to such a degree that the circulation of blood to the retina and thus also the function thereof is impaired, wherein this functional impairment may lead to the eye disease known as glaucoma and may result in irreversible blindness in the affected eye.

For improving and maintaining the anatomical drainage of the aqueous humor, support elements embodied as elongated tubes with holes distributed over the surface thereof, or as elongated, tube-shaped net meshes or the like may be used as described in the publications (EP 0 898 947 A2 and EP 1 125568 A2), and can be inserted (or released) into the Schlemm's canal, which is exposed by creating an incision and folding back a scleral flap, and opened by injecting a high-viscosity medium. The elongated support elements are designed to facilitate the anatomically natural outflow of the circulating and permanently renewable aqueous humor from the anterior chamber through the trabecular meshwork and into the lumen of the circular Schlemm's canal, and from there into the blood circulation via the episcleral venous system.

In some arrangements a T-shaped implant which is attachable by means of a plate to the sclera in which an incision is made may be used as described in the document (US 2004/0210181 A1), and comprises a proximal piece of tubing that can be inserted operatively directly into the anterior chamber or through the trabecular meshwork into the anterior chamber, as well as two distal tube members which are arranged opposite each other on the piece of tubing and are insertable into the exposed Schlemm's canal. In order to avoid elevated intraocular pressure (IOP) in the case of a pathologically closed trabecular meshwork, with the implant which is designed to enable drainage, the constantly renewable aqueous humor is guided on an artificially created pathway by the proximal piece of tubing inserted into the anterior chamber, via the distal tubes directly into the Schlemm's canal, and from there into the blood circulation of the eye through the episcleral venous system. Further implants for the treatment of glaucoma, may be used as described in both documents (US 2005/0192527 A1 and 2007/0088432 A1), and which are either formable into an approximate T-shape with a thermal or mechanical shape memory effect or are constructed in an approximate T-shape without any shape memory effect.

These exemplary implants are each insertable with a proximal tube either directly or operatively through the trabecular meshwork into the anterior chamber and with two distal tubes arranged oppositely on the proximal tube into the Schlemm's canal, so that the constantly renewable aqueous humor is also transported along an artificially created pathway from the anterior chamber directly into the Schlemm's canal and from there into the blood circulation of the eye via the episcleral venous system.

The generally known canaloplasty method further offers the option of a circumferential dilation, in which the Schlemm's canal is expanded circularly by means of an inserted, flexible microcatheter and at the same time or subsequently a high-molecular-weight viscoelastic substance is injected by means of a “microscrew”. The microcatheter is then retracted again, and the circular Schlemm's canal is stretched towards the anterior chamber by suitable means, with a surgical thread, for example, resulting in an expansion of the trabecular meshwork and greater permeability with favourable transtrabecular drainage of the aqueous humor.

In exemplary arrangements various production steps and etching methods may be used. For example, Application DE 100 07 425 A1 includes a description of a method for producing an implant based on a photoresist. Here, the selectivity of the subsequent etching process is enabled a by chemical reaction in the photoresist. The method is used in the semiconductor industry when silicon chips are exposed to light.

Other exemplary production method steps may be used. For example EP 2 473 311 describes a method in which an opaque base material is used that facilitates material removal with a laser.

However, some known production methods share the disadvantage that they are relatively expensive for mass production, and the implants cannot be manufactured with a process reliability that assures consistent quality.

Exemplary arrangements provide a production method for producing an implant configured for insertion in the Schlemm's canal and also new implant configurations, with which such implants may be produced rapidly, inexpensively and reproducibly.

Useful results are provided in accordance with the methods recited in the claims hereof.

Alternatively a suitable implant may also be created additively in layers from powdered material by melting, or material dissolved in fluid is solidified by local heat input in the focus area in such a way that the implant is formed. It is useful when forming the implant by melting if a continuous wave laser is used, the radiation from which is substantially absorbed by the material. For example, a CO₂ laser with a wavelength of 10 μm is suitable for this purpose.

It is useful if the implant is manufactured from a biocompatible material that is produced either additively or subtractively with laser radiation.

In producing an exemplary implant using SLE (Selective Laser Etching), the selectivity is achieved by non-linear absorption in a material that is intrinsically transparent for the wavelengths used (glass, sapphire, visible light). In the process, the absorption is enabled by exceeding a threshold intensity, which is situated at about 10¹⁰-10¹³ W/cm² depending on the material and wavelength. At these intensities, the refractive index of the transparent material of the provided implant blank is non-elastic, that is to say it is altered permanently, and thus for the fluid etching media subsequently used (e.g., KOH) the etchability/etching rate are also altered compared with the unchanged material. Accordingly, this is a completely different process from the methods used hitherto for achieving the requisite selectivity.

Application DE 10 2015 115 958 A1 describes a special composition of glass constituents, which influence a cell population on the implant when used in some exemplary arrangements depending on the use case.

The exemplary subtractive process described has the advantage that local, sufficiently high intensities are able to alter an implant blank that is provided in such a way that the material can be removed from the blank in the regions and areas of the blank that are subjected to radiation in this manner, thereby giving rise to cavities in those places (e.g., by SLE=Selective Laser Etching).

In this context, the irradiated material can be eliminated directly or in a second, subsequent process, only at the sites where the exposure to the laser has altered the material correspondingly in such a manner that the second (etching) process removes the material, in a fluid etching step for example.

The implant of exemplary arrangements may be produced from various materials. Glass, sapphire or plastic are advantageous materials for the implant. A glass fibre or hollow glass fibre, which is preferably processed further with the SLE method, may also serve as starting material. In such case, the glass fibre may be shortened to a specific length before or after the treatment.

The wavelength of the laser radiation used in exemplary methods may have a value between 150 nm and 1800 nm, preferably 1064 nm or 532 nm or 334 nm or 266 nm.

Pulsed laser radiation is preferably used for laser etching.

The exemplary focal radius of the laser radiation should be between 1 μm and 100 μm, preferably about 5 to 10 μm. Useful values for the focal radius _{rF} are in the range from 0.5 to 5 μm and in particular from 1 to 3 μm.

The pulse energy of the laser radiation may be in a range between 0.3 μJ and 3 μJ. Useful pulse energies have values in the range from _{Ep}=0.1-1 J, for example, and in particular from 0.3 to 0.8 μJ. The pulse duration T may have a value from 0.5 to 5 ps and in some particular arrangements 0.8 to 2 ps.

The pulse frequency of the laser radiation may be in a range from 1 kHz to 1 MHz, wherein average output of the laser should be between 1 mW and a few Watts. Useful intensities I are in the range from 10¹² to 10¹⁴ W/cm² and in particular arrangements about 10¹³ W/cm². For the repetition rate v REP, values between 10 kHz and 1 MHz and in particular arrangements between 50 kHz and 500 kHz are suitable. The average output P_{quer} then has a value for example of 1 mW to 1 W and in particular arrangements 100 to 500 mW. Ultimately, in some arrangements they may be for example approximately 10¹¹ W/cm²×10⁻¹¹ s×(5 μm)².

In some exemplary arrangements the implant may be polished so that it has a smooth surface, with no notches which might make it more likely to break.

Laser radiation may be used for polishing the implant. In this case, for example a highly absorbable CO₂ laser beam is suitable. The polishing should have the effect of smoothing the surface of the implant. This may provide the effect of preventing or at least reducing the notching effect, to increase fatigue strength under cyclic bending stresses and improve breaking resistance as well as providing greater elasticity and lower risk of breakage. A smooth surface also makes it easier to introduce the implant into the body, such as in particular the eye or the Schlemm's canal.

Alternatively or additionally, the implant may also be polished using an ion etching process in an ion etching process step.

An advantageous alterative process step provides that the implant is polished by Rapid Thermal Annealing (RTA). This is a furnace melting process at high temperatures and with short heating times. In some exemplary arrangements, very rapid heating is achieved with halogen lamps, followed immediately by very fast cooling. The heating may also be performed with a laser instead of or in addition to halogen lamps. This process may be carried out in a vacuum or in an inert gas atmosphere to avoid oxidation processes. In this context, time intervals for the heating and cooling and the temperatures used are harmonized with the special implant and the material thereof.

In some arrangements it is useful if the implant produced via the method has a hollow-cylindrical form and is liquid-permeable on opposite sides of the cylinder lateral surface. In such a case, the cylinder in cross-section may be elliptical or conical in shape.

If single openings to the hollow cylinder shape body are provided, the maximum cross-section thereof should be in the range from 10 μm to 50 μm. Of course if the openings are configured in a spiral, for example, the openings may also be larger. Moreover, openings with shapes other than circles or ovals are also possible.

The overall length of the exemplary implant may be from 0.1 cm to 5 cm, but in particular arrangements from 2 to 3 cm.

The diameter of the implant in the exemplary arrangements has a value between 30 μm and 300 μm, and in some arrangements about 100 μm. The exemplary implant may have a lattice-like structure, which extends longitudinally or also transversely to a longitudinal axis of the implant.

In some exemplary arrangements that the implant has a curved longitudinal axis.

An exemplary implant can be inserted in the Schlemm's canal of an eye at least as far as a quarter of the circumferential direction of the lumen of the circular Schlemm's canal. An insertion length of one quarter is advantageous. However, in some arrangements the implant may also be shorter, and may only be insertable into any fraction of the circumferential direction of the lumen.

In some exemplary arrangements the implant has a base body and a tip at one end of the body. This tip does not have to be sharp, and in some arrangements may also have a domed shape, for example. The radius of curvature of the tip in some arrangements may have a value of half the diameter of the implant and is thus in some arrangements is between 15 μm and 150 μm. The tip in some arrangements may also be asymmetrical. This makes it easier, for example in arrangements that have a configuration such as that of a dolphins snout, to insert the implant into a curved Schlemm's canal.

If it is desired to avoid having to mount or connect the tip separately, the tip may be produced in a single part with the base body.

In some exemplary arrangements the implant has struts which extend diagonally to the longitudinal axis and to the circumferential direction of the circumferential surface of the cylinder. In this way, a lattice structure may be created, in which a strut may also run transversely through a hollow cylinder.

In some exemplary arrangements the struts are helical, spiral or screw-shaped, and form at least part of the cylinder lateral surface. The formation of a helical circumferential line can make it possible to screw the implant into the eye along the circumferential line. In such a case, the struts may be designed as single or multi-thread helixes, as spirals with an open or porous filled core, or as screws.

In some exemplary arrangements the implant is opaque. For example, a coating or color may be applied in a process step after the implant has been manufactured, or a coloration may even be imparted during polishing, which enables visibility during a gonioscopy (chamber angle reflection with special lenses). This may also make it easier to reopen clogged openings in the implant, with a laser such as a Yag laser, if permeability disturbances are detected. A coloration such as a red end, may also serve as a mark to identify the manufacturer.

In some exemplary arrangements the implant has a longitudinal axis and is constructed symmetrically about said axis.

In order to facilitate the insertion of the implant in a lumen of a Schlemm's canal for example, some exemplary implant arrangements have a longitudinal axis and the cylinder lateral surface includes radially inward contact surfaces, which are arranged at an acute angle to the longitudinal axis. When such an implant is pushed into a lumen, the inclined contact surfaces press radially inwardly protruding subregions of the lumen outwards, while the implant is pushed into the lumen and in the lumen.

In some exemplary arrangements in which the implant has a cylindrical body a rod-shaped, spiral-shaped or sleeve-shaped insertion aid that can be connected detachably to the implant. An exemplary rod-shaped or spiral-shaped insertion aid may be screwed or pushed into a hollow cylindrical implant and the implant may be held in a sleeve-shaped insertion aid while it is inserted in the eye. During the insertion of the implant, it is connected to the insertion aid, and afterwards the insertion aid may be removed without changing the position of the implant. However, the insertion aid may also be used to push the implant into Schlemm's canal by positioning the insertion aid behind the implant.

In some exemplary arrangements the implant is spiral in shape, and the insertion aid has a correspondingly spiral-shaped counterpart, so that the implant can be screwed onto the insertion aid and, after insertion, the insertion aid may be unscrewed from the implant by rotating.

In some exemplary arrangements the spiral-shaped counterpart of the insertion aid may form an essentially smooth cylinder lateral surface with the cylinder lateral surface of the implant, when the implant is screwed on.

In some exemplary arrangements, the insertion aid may also be longer than the cylinder lateral surface, and it may have a handle which makes it possible to move and preferably even rotate an end of the insertion aid located inside the implant.

In the following discussion a medical procedure for inserting an implant produced via the exemplary methods will be explained. Of course these described approaches are exemplary and other approaches, arrangements and processes may be used.

### BRIEF DESCRIPTION OF DRAWINGS

FIG. 1 shows an enlarged, schematic representation of a longitudinal section through an eye.

FIG. 2 shows a schematic representation of a front view of the eye with a parabolic incision in the sclera and an opened scleral flap.

FIG. 3 shows a section of the eye along the section line A-A shown in FIG. 2 on a larger scale with partially exposed Schlemm's canal.

FIG. 4 shows a section of the eye, shown on a larger scale, with an injection probe inserted in the Schlemm's canal.

FIG. 5 shows a partial section of the exposed Schlemm's canal according to FIG. 4, on a larger scale, with an exemplary implant inserted into the lumen thereof and released.

FIG. 6 shows an eye section of the eye during insertion of the exemplary implant.

FIG. 7 shows an enlarged section from FIG. 6 without an implant.

FIGS. 8 to 19 show different exemplary implant arrangements.

FIG. 20 shows an exemplary implant with contact surfaces.

### DETAILED DESCRIPTION

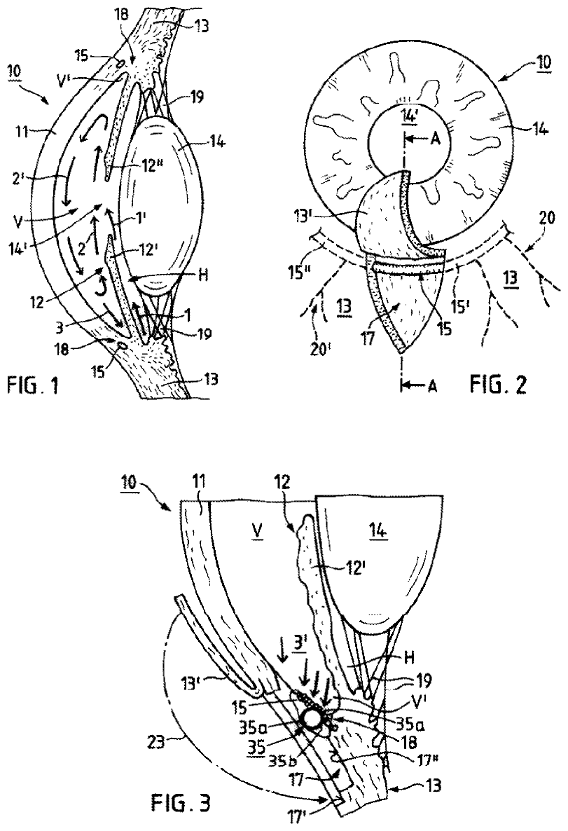

At this point, it is noted that FIGS. 1 to 5 each show a section of the eye for a better understanding of the use of exemplary arrangements as they relate to glaucoma surgery. Moreover, identical parts are provided with the same reference symbols in the individual Figures and in the following description.

FIG. 1 shows, in the representation already known from the publication EP 0 898 947, a schematically represented vertical section of the front section of an eye 10, in which the cornea 11, the iris 12 with the two areas 12V and 12W, the dermis 13 (sclera), the lens 14 with the pupil 14′, the zonule fibres 19, the circular Schlemm's canal 15 (sinus venosus sclerae) and the trabecular meshwork 18 (trabeculum comeo sclerale) in front of Schlemm's canal 15 are visible. As shown schematically in FIG. 1, in a healthy eye the outflow of the circulating and constantly renewing aqueous humor (humor aquosus) takes place from the posterior chamber H to the anterior chamber V according to the arrows 2 and 2′, and at the chamber angle V′ (angulus iridocomealis) it is transported away in the direction of arrow 3 via the trabecular meshwork 18 into the lumen of the circular Schlemm's canal 15, and from there returns to the blood circulation via the episcleral venous system, not shown in FIG. 1. As was stated previously, in the case of pathological conditions of the affected eye, a continuous outflow of the aqueous humor formed by the epithelium of the ciliary body and constantly renewing itself is often no longer assured. Schlemm's canal 15 may become closed in such a way that the outflow of aqueous humor is obstructed or extensively blocked, with the result that the intraocular pressure rises to such an extent that the blood flow to the retina and consequently the function thereof is limited to the point that the affected eye becomes blind.

FIG. 2 shows a representation of the eye 10, also known per se from the publication EP 0 898 947, in a schematic front view, and the lens 14 with the pupil 14′, a section of the dermis 13, a portion of the Schlemm's canal 15 as well as a portion of the natural canal system 20, 20′ (aqueous humor canal system) associated therewith can be seen. The Schlemm's canal 15, shown partially and schematically in FIG. 2, extends circumferentially through an angle of 360° and runs circularly around the lens 14. In a microsurgical operation, a lamellar incision is made in the dermis 13 in a manner known per se as represented schematically in FIG. 2, and after a section of the dermis (not further shown) has been severed, the outer flap-like section 13′ is folded back and held in place by means not shown for the further operative intervention. The lamellar incision forms a scleral bed with designation numeral 17 in the region of the exposed Schlemm's canal 15, the scleral bed being closed again with the section 13′ (scleral flap) that can be folded down in the direction of arrow 23 (FIG. 3) upon completion of the surgical intervention, for example after the insertion and release of an elongated implant. In a further variant of the microsurgical intervention, however, there is also the possibility that the trabecular meshwork 18 (FIG. 3) in front of the circular Schlemm's canal 15 is opened at least partially in circular manner to enable the insertion and release of the implant with a cutting instrument or the like that is introduced into the anterior chamber V in a way that is not shown.

FIG. 3 shows the portion of the eye 10 represented according to the line A-A marked in FIG. 2 in cross-section and on a larger scale, showing the cornea 11, the first region 12′ of the iris 12, the dermis 13 with the opened scleral flap 13′, the lens 14, the zonule fibres 19, the posterior chamber H and the anterior chamber V, with the chamber angle V′, the trabecular meshwork 18 and the Schlemm's canal 15 with the implant 35 arranged therein.

The Schlemm's canal 15 is oriented circularly around the cornea and, as shown schematically and on a larger scale in FIG. 3, extends substantially along the trabecular meshwork 18 and has a roughly oval cross-sectional profile and a form that substantially tapers from the one end in the region chamber angle V′ towards the other end. The trabecular meshwork is, so to speak, the interior wall of Schlemm's canal with the eye. The trabecular meshwork separates the inside of the eye from the canal and ensures that the water does not drain without resistance. The scleral bed 17 exposed by the incision, with the inner surface 17″ and the contact surface 17′ for the scleral flap 13, are also visible in FIG. 3.

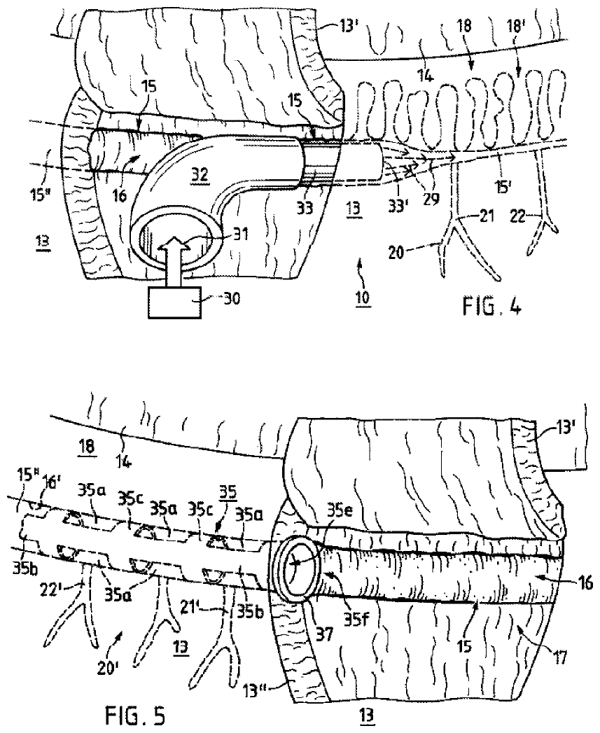

In FIG. 4, a tubular probe 33 arranged on a connecting member 32 is introduced into the lumen 16 of the exposed Schlemm's canal 15 in order to enlarge the Schlemm's canal 15 in a manner known per se. The connecting member 32 is connected to a schematically represented injection device 30 via a feed line, which is not shown. With the injection device 30, a hydrophilic liquid 29 for example is injected into the Schlemm's canal 15 in the direction of arrow 31 via the tubular probe 33′ which is provided at the distal end with at least one outlet opening 33′, which consequently causes a circumferentially oriented section 15′ of the Schlemm's canal 15 to be expanded hydraulically.

The section 15″ of the Schlemm's canal 15 that is opposite the already treated section 15′ can also be treated in the same way and expanded hydraulically in a circular direction in a manner known per se with a mirror-inverted probe inserted into the Schlemm's canal 15.

FIG. 4 also shows the trabecular tissue 18 (trabecular meshwork) in front of the Schlemm's canal 15 with the schematically represented trabeculae 18′ and the canal system 20 with the small channels 21 and 22.

During the expansion of the Schlemm's canal 15 described above, openings (not shown) that may arise in the wall are opened and stretched at the same time with the hydrophilic fluid 29, with the result that these collectors are also reactivated and ensure the outflow of the aqueous humor. Instead of the hydrophilic fluid, a suitable, biologically compatible gaseous medium or also a mixture of the hydrophilic fluid and the gaseous medium can also be used to expand Schlemm's canal.

As is represented schematically in FIG. 5, following the hydraulic or pneumatic expansion and to optimize permanent permeability and circulation of the aqueous humor, an implant 35 produced according to the previously described methods is introduced into the lumen 16 of the circular Schlemm's canal 15. The exemplary implant 35 consists of an elongated, flexible tube 36 and is preferably made of biocompatible, elastic material and is introduced into the lumen 16 of Schlemm's canal 15 by appropriate means, not shown in detail, for example by means of a probe (inserting instrument) or the like.

FIG. 5 further shows a portion of the exemplary implant 35 which is introduced into the Schlemm's canal 15, and which is arranged detachably on the probe (inserting instrument) with the proximal end (nearest to the inserting instrument) (not shown). On the other, distal end (farthest from the inserting instrument), the implant 35 is provided with an abutment collar 37 that rests against the inside 13V of the dermis 13″ and has an opening 35f. The implant 35 introduced into the lumen 16 extends from one inner side 13″ of the exposed Schlemm's canal 15 in a manner not shown in detail, up to at least a quarter, half, three-quarters or preferably the entire circumferential direction as far as the opposite inner side of the lamellar incision (FIG. 2). In a variant not shown, the possibility also exists of introducing substantially semicircularly curved implants 35 into the exposed circular Schlemm's canal 15 from both the one side and from the other, opposite side of the lamellar incision. With the implant 35, the lumen 16 of the circular Schlemm's canal 15 is supported and kept open permanently.

FIG. 5 also shows the scleral bed 17 that is formed by the lamellar incision between the two opposite inner sides 13″, which when the scleral flap 13′ is folded down and placed on the parabolic support surface 17′ and is appropriately sutured to the dermis 13 forms a subscleral space or collecting basin (reservoir) for the aqueous humor. The scleral bed 17 is connected to the interior space 35e of the implant 35 via the two oppositely arranged openings 35f (only one opening 35f is shown) of the implant.

FIG. 5 further illustrates a portion of the exemplary implant 35 introduced into the Schlemm's canal 15, which lies against the inner wall 16′ of the lumen 16 in supporting manner with ring parts 35c arranged at a distance from one another. As shown in FIG. 5, openings or apertures 35a arranged between the individual ring parts 35c each form a direct connection, which is permanently kept open between the trabecular meshwork 18 and the individual small channels 21′ and 22′ of the channel system 20′, so that the natural transtrabecular outflow of the aqueous humor from the anterior chamber V via the trabecular meshwork 18 into the circular Schlemm's canal 15 and into the interior space 35e of the implant 35, and from there via the episcleral venous system into the blood circulation is assured.

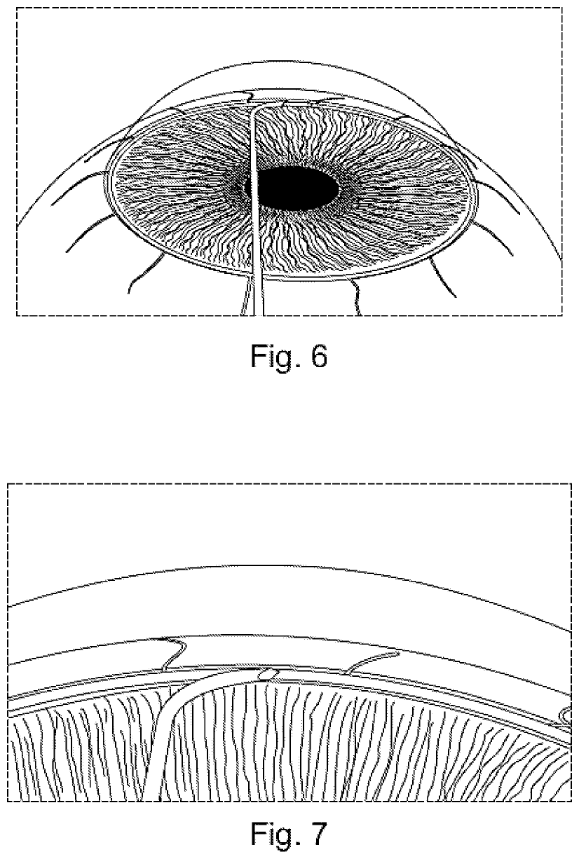

As an alternative to the “ab externo” route into the eye via the scleral access, the implant may also be inserted “ab interno” tangentially to the Schlemm's canal through a Clear Cornea access. This would allow minimally invasive glaucoma surgery with an incision of about 2 mm and less. For this purpose, the Schlemm's canal is opened at the point opposite the incision by a minimal opening of the trabecular meshwork and from here the exemplary implant is pushed tangentially into the possibly pre-stretched Schlemm's canal. This is shown in FIGS. 6 and 7. For this purpose, an access is made through the clear cornea, as in the cataract operation, which leads tangentially to Schlemm's canal. Ab interno, the Schlemm's canal is punctured and the implant introduced through this opening into Schlemm's canal. For this purpose, the implant can be inserted with a stylet or simply pushed into the canal. The implant should be sufficiently rigid or firm in its longitudinal extension to enable it to be pushed into the canal, and it should be elastic or flexible enough transversely to the longitudinal extension to be able to follow the canal.

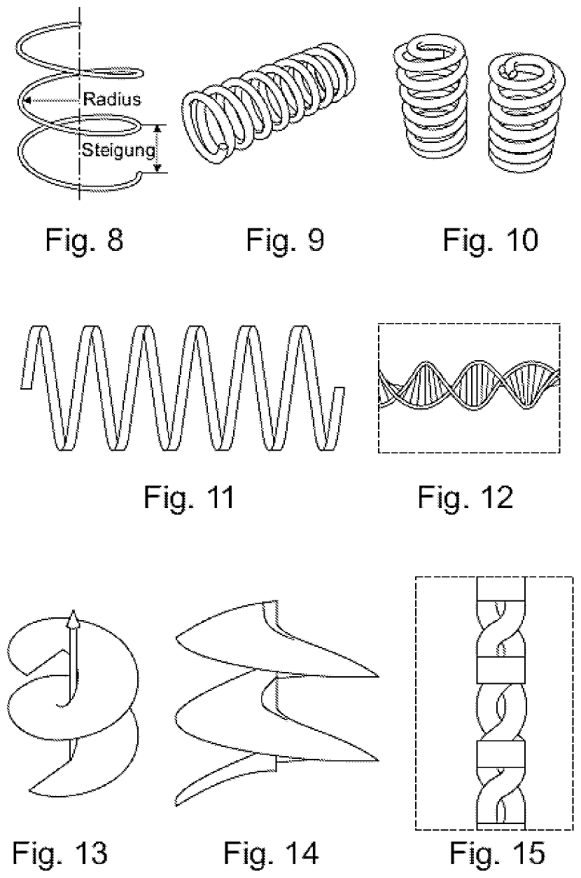

FIGS. 8 to 19 show different exemplary arrangements of implants produced in accordance with the method steps previously described, wherein the implants have a longitudinal axis and are symmetrical along said longitudinal axis. When configured as a screw, the implant may be configured as a helical spring. The helical spring is configured in such a way that when the implant is pushed into Schlemm's canal the coils are sufficiently rigid not to be pushed and compressed into a block, and the spring coils are flexible enough to be pushed into a curved canal as well, and in so doing to adapt to the shape of a curved channel.

FIG. 10 shows how one end of an exemplary implant may be configured. However, in some arrangements it is useful if the end is dome-shaped or designed with a configuration like a dolphin's snout, so that the implant can be pushed into a channel easily.

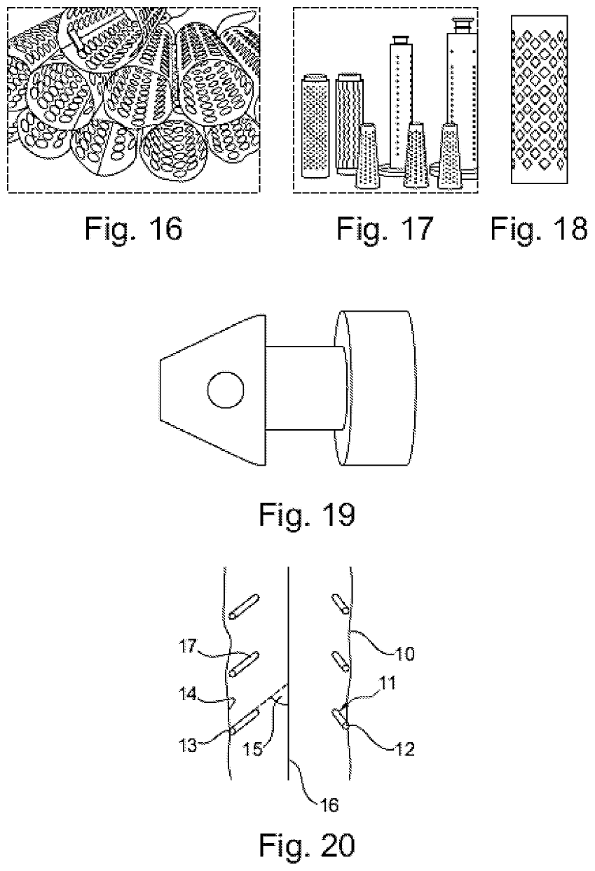

In the case of an exemplary implant having a screw shape, the base body may be in the form of a band and wound around a longitudinal axis as shown in FIG. 11. However, as shown in FIG. 12, a band may also be rotated about a longitudinal axis to form a screw. The edges of this band then form a double helix. Further screw shapes for exemplary implants are shown in FIGS. 13 and 14, wherein a cylindrical body extends along a central axis of these screw shapes and is hollow. This is shown in exemplary implant configuration of FIG. 15, for example, in which a double helix is held in place by annular shoulders. Further exemplary implant configurations shown in FIGS. 16 to 18 resemble a hair curler or yarn dye bobbins.

However, as shown in FIG. 19, an exemplary implant may also have a shape which is known as iStent™ inject from the Glaukos company.

FIG. 20 shows in a longitudinal section through an exemplary implant 11 having a helical spring configuration and inserted into a lumen 10, the points 12 which are in contact with the lumen 10. In the case of a helical spring with round cross-section, the regions 12 of the helical spring 13 that come into contact with the lumen 10 are already rounded. Consequently, these rounded areas already form a cylinder lateral surface with contact surfaces 14 which are bent radially inward and are arranged at an acute angle 15 to the longitudinal axis 16 of the implant 11. FIG. 20 shows in exaggerated form, exemplary contact surfaces 17 which make it easier to push the implant 11 into the lumen 10.

Thus the exemplary arrangements described herein provide improved operation and capabilities, eliminate difficulties encountered in the use of prior methods and devices, and attain the useful results that are described herein.

In the foregoing description, certain terms have been used for brevity, clarity and understanding. However no unnecessary limitations are to be implied therefrom because such terms are used for descriptive purposes and are intended to be broadly construed. Moreover the descriptions and illustrations herein are by way of examples and the new and useful features, relationships and methods are not limited to the exact features, relationships and method steps that have been shown and described.

It should be understood that the features, relationships and method steps associated with one arrangement can be combined with features, relationships and method steps from another arrangement. That is, features, relationships and method steps various arrangements can be combined into other arrangements. The new and useful scope of the disclosure is not limited only to the arrangements that have been shown and described.

Having described features, discoveries and principles of the exemplary arrangements, the manner in which they are carried out, constructed and utilized, and the advantages and useful results attained, the new and useful features, devices, methods, approaches, elements, arrangements, parts, combinations, systems, processes, equipment, operations, methods and relationships are set forth in the appended claims.

1.-30. (canceled)

31. A method comprising:
producing an implant configured to be implanted into a Schlemm's canal of an eye including the steps of:
a) providing an implant blank, wherein the implant blank is comprised of material that is permeable to laser radiation,
b) subjecting at least one region of the implant blank to laser radiation,
c) subsequent to (b), removing material from the at least one region via fluid etching.

32. The method according to claim 31
wherein in (a) the implant blank is comprised of glass or sapphire.

33. The method according to claim 31
wherein in (a) the implant blank is comprised of plastic.

34. The method according to claim 31
wherein in (b) the wavelength of the radiation is between 150 nm and 1800 nm.

35. The method according to claim 31
wherein in (b) the wavelength of the radiation is at least one of 1064 nm, 532 nm 334 nm and 266 nm.

36. the method according to claim 31
wherein in (b) the laser radiation includes pulsed laser radiation.

37. The method according to claim 31
wherein in (b) the focal radius of the laser radiation is between 1 μm and 100 μm.

38. The method according to claim 31
wherein in (b) the focal radius of the laser radiation is between 5-10 μm.

39. The method according to claim 31
wherein in (b) the laser radiation includes pulsed laser radiation with a pulse energy value of 0.3 μJ to 3 μJ.

40. The method according to claim 31
wherein in (b) the laser radiation includes pulsed laser radiation with a pulse frequency of the laser radiation of from 1 kHz to 1 MHz.

41. The method according to claim 31, and further comprising:
(d) subsequent to (c), polishing the blank.

42. The method according to claim 31, and further comprising:
(d) subsequent to (c), polishing the blank using at least one of
laser radiation,
ion etching, and
rapid thermal annealing.

43. The method according to claim 31
wherein subsequent to (c) the implant has a hollow cylindrical shape including a cylindrical lateral surface that is fluid permeable.

44. The method according to claim 31
wherein subsequent to (c) the implant has a hollow cylindrical shape including a lateral cylindrical surface that is fluid permeable by including openings therein with a maximum cross-section of from 10 μm to 50 μm.

45. The method according to claim 31
wherein subsequent to (c) the implant has a hollow cylindrical shape with an overall length of 0.1 cm to 5 cm and including a lateral cylindrical surface that is fluid permeable.

46. The method according to claim 31
wherein subsequent to (c) the implant has a hollow cylindrical shape having a diameter of from 30 μm to 300 μm and including a lateral cylindrical surface that is fluid permeable.

47. The method according to claim 31
wherein subsequent to (c) the implant has a hollow cylindrical shape that has a curved longitudinal axis and that includes a lateral cylindrical surface that is fluid permeable.

48. The method according to claim 31
wherein subsequent to (c) the implant has a hollow cylindrical shape with a lateral cylindrical surface that extends along a longitudinal axis and that includes struts which extend diagonally to the longitudinal axis and in a circumferential direction of the lateral cylindrical surface.

49. The method according to claim 31
wherein subsequent to (c) the implant has a hollow cylindrical shape with a lateral cylindrical surface that extends along a longitudinal axis and that includes struts having a helical, spiral or screw shape, which struts form at least a part of the lateral cylindrical surface.

50. The method according to claim 31
wherein subsequent to (c) the implant has a hollow cylindrical shape with a lateral cylindrical surface that extends along a longitudinal axis and that includes radially inward contact surfaces which are arranged at an acute angle to the longitudinal axis.

51. The method according to claim 31 and further comprising:
subsequent to (c), rendering the implant opaque.

52. The method according to claim 31
wherein subsequent to (c) the implant has a hollow cylindrical shaped body with a lateral cylindrical surface that extends along a longitudinal axis and that includes an asymmetrical tip on one end of the body.

53. The method according to claim 31
wherein subsequent to (c) the implant has a hollow cylindrical shape body with a lateral cylindrical surface that extends along a longitudinal axis that is configured to be engaged with an insertion aid to facilitate placement of the implant within the Schlemm's canal, which insertion aid is configured to be screwed into and unscrewed from the body.

54. The method according to claim 31
wherein subsequent to (c) the implant has a hollow cylindrical shape body with a lateral cylindrical surface that extends along a longitudinal axis, wherein the implant is configured to be inserted into a circular lumen of the Schlemm's canal at least one fourth of the circumferential length of the lumen.
